(19) United States
(12) Patent Application Publication
(10) Pub. No.: US 2012/0213337 A1
(43) Pub. Date: Aug. 23, 2012

(54) BITE WING FOR DENTAL SENSORS

(76) Inventor: Gerald Gestetner, Montreal (CA)

(21) Appl. No.: 13/400,547

(22) Filed: Feb. 20, 2012

Related U.S. Application Data
(60) Provisional application No. 61/457,299, filed on Feb. 22, 2011.

(51) Int. Cl. A61B 6/14

(52) U.S. Cl. 378/189

(57) ABSTRACT

A positioning bite tab for a dental sensor, said positioning bite tab comprising: a central section and two wings extending substantially opposed to each other from said central section, each of said wings including a cushioned section.

### FIELD OF THE INVENTION

The present invention relates to the general field of radiography, and is more specifically concerned with a positioning bite tab for dental sensors, for example for a dental digital sensor.

### BACKGROUND

Digital x-ray sensors are used by dentists as a replacement to traditional X-ray methods. The sensor is covered by an infection control barrier and then placed in the patient's mouth in the desired area. The purpose of the bite wing is to have the sensor remain in the proper location through out the X-ray process and not be moved around by the mouth. However, currently used bite wings are typically relatively uncomfortable when inserted in the mouth and are relatively difficult to operate so that the radiographic sensor is optimally positioned.

Against this background, there exists a need in the industry to provide an improved positioning bite wing for dental sensors. An object of the present invention is therefore to provide such a positioning bite wing for dental sensors .

### SUMMARY OF THE INVENTION

In a broad aspect, the invention provides a positioning bite tab for a dental sensor, said positioning bite tab comprising a central section and two wings extending substantially opposed to each other from said central section, each of said wings comprising a cushioned section. In some embodiments of the invention, the cushioned section includes a gas filled cavity or bladder forming a cushion. For example, the gas is air, but other gases are within the scope of the invention.

Advantageously, the proposed bite wing has been designed with optimal patient comfort and superior positioning in mind. Also, the proposed bite wing is relatively simple to use. In embodiments of the invention in which the gas filled cavity is provided, the presence of the gas allows proper bite of the bite wing both for patients who can bring their teeth in contact with each other when closing their jaw and in patients in which a gap is present between teeth that are in register with each other even when their jaw is closed. This is due to the fluid nature of the gas that can flow to any suitable location in the cavity. Also, in some embodiments of the invention, the cavity is defined by a bladder that is resiliently deformable to accommodate displacement of the gas due to teeth biting the cushioned section.

In another broad aspect, the invention provides a bite wing for a dental sensor, said bite wing comprising a biting section and an strap extending from said biting section, said biting section being provided with a cushion.

Advantageously, the proposed bite wing has been designed with optimal patient comfort and superior positioning in mind, is relatively simple to use. and is compatible with many commercially available digital sensor sizes.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawing:

FIG. 1, in a front elevation view, illustrates a positioning bite tab in accordance with an embodiment of the present invention;

FIG. 2, in a back elevation view, illustrates the positioning bite tab shown in FIG. 1;

FIG. 3, in a side elevation view, illustrates the positioning bite tab shown in FIG. 1, the positioning bite tab being shown in an open position;

FIG. 4, in a side elevation view, illustrates the positioning bite tab shown in FIG. 1, the positioning bite tab being shown in a closed position;

FIG. 5, in a perspective view, illustrates the positioning bite tab shown in FIG. 1;

FIG. 6, in a front elevation view, illustrates a bite wing in accordance with an alternative embodiment of the present invention;

FIG. 7, in a back elevation view, illustrates the bite wing shown in FIG. 6;

FIG. 8, in a side elevation view, illustrates the bite wing shown in FIG. 6, the bite wing being shown in a closed position;

FIG. 9, in a top plan view, illustrates the protective bite wing shown in FIG. 6, the bite wing being shown in the closed position; and

FIG. 10, in a side elevation view with a partial cross-section, illustrates a bite wing in accordance with another alternative embodiment of the invention.

### DETAILED DESCRIPTION

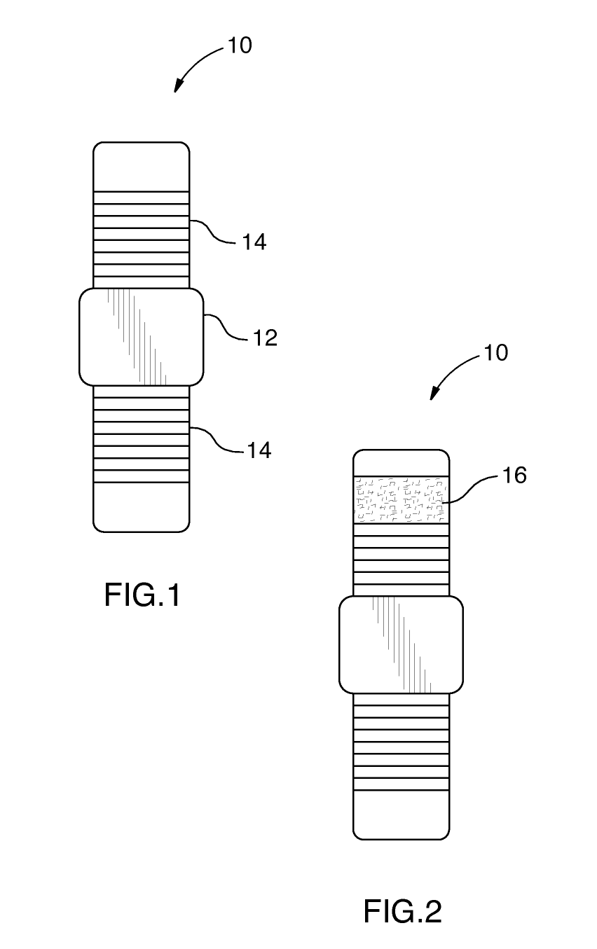

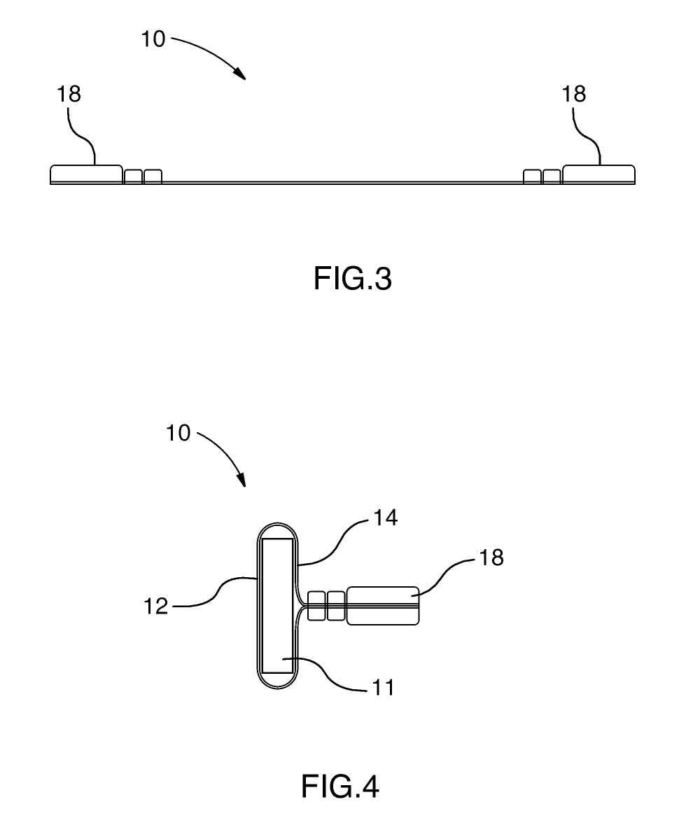

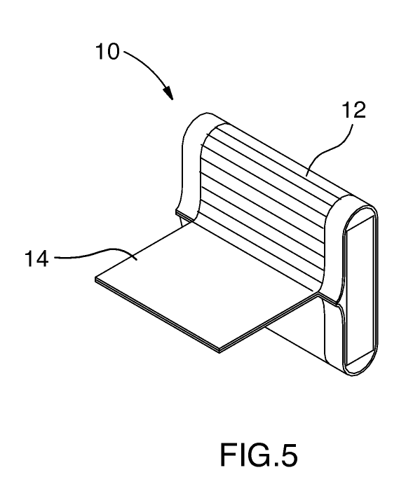

Referring to FIGS. 1 to 5, there is shown a bite wing 10 in accordance with the present invention. The bite wing 10 is substantially elongated and designed with a wider central section 12 where a sensor (not shown in FIG. 1) is to be placed and two wings 14 extending substantially opposed to each other therefrom. After positioning the sensor 11 in the central section 12, the next step in use is to fold the wings 14 over the sensor 11, remove the double-sided adhesive's liner 16 part of the wings 14 (seen in FIG. 2) and press the two adhesive-covered sides of the wings 14 together forming a tight grip on the sensor 11. The double-coated adhesive liner 16 is located in a substantially central position on the wings 14 which allows it to form a tight grip on most popular size digital sensors on the market. This positioning allows a part of the adhesive 16 to attach to the sensor and the rest to attach the to wing 14 ends. On the end of each strap is ¾″ piece of relatively soft foam 18, which come together once the bite wing 10 is fixed on the sensor to form a soft and durable area for the patient to bite into. The foam 18 is also placed in a central location relatively to the wing 14 where it can be fully used in the bitetab portion when a small sensor 11 is being used and can fold along with the strap when a larger sensor is being used. The material used for the actual body is, for example, a 12 point vinyl which is relatively durable and does not add significant bulk to the sensor. The foam 18 is for example a soft vinyl foam approximately ⅛″ thick, (¼″ thick when combined both sides) that is welded to the vinyl body. This bite wing 10 is relatively comfortable to use because of its soft and unbulky materials.

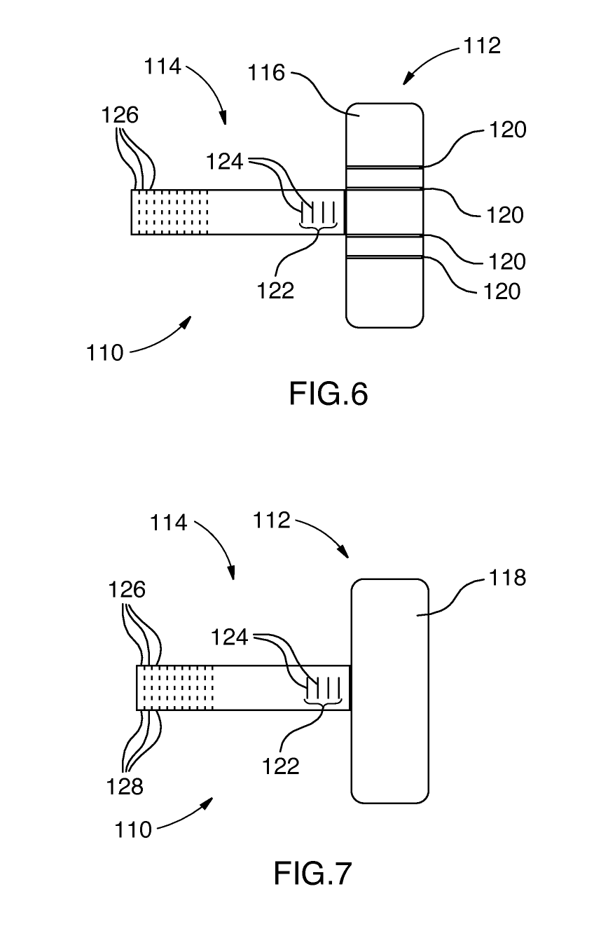

Referring to FIGS. 6 to 9, there is shown a bite wing 110 in accordance with the present invention, with numerical values for dimension given by way of example only. The bite wing 110 includes a biting section 112 and a strap 114 that attaches the bit wing 10 to a sensor (not shown in the drawings). The strap 114 is wrappable around the width of the sensor to allow the sensor wire, which runs out of the bottom of the sensor, to exit the bite wing 110. The strap 114 and the biting section 112 are together generally T-shaped and extend substantially perpendicularly to each other.

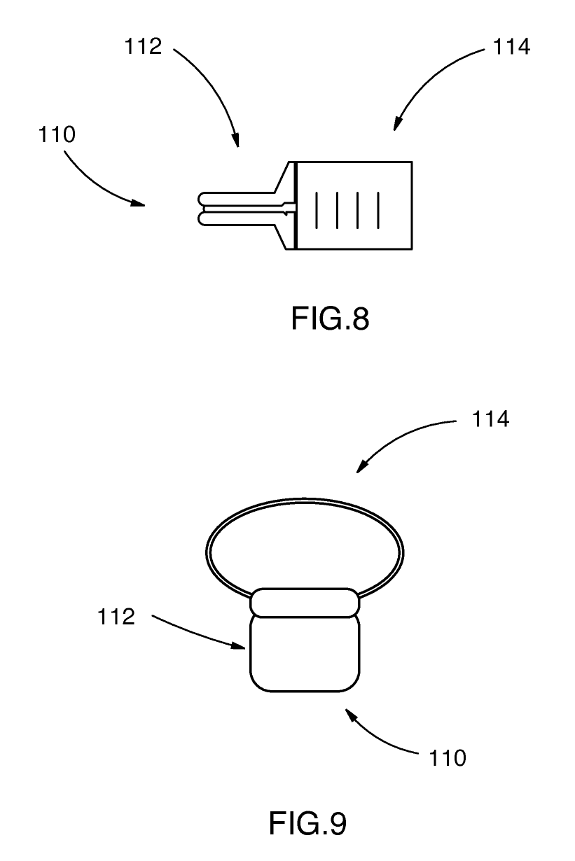

The biting section 112 is provided on one side with a cushion 116, as seen in FIG. 6, and one the opposite side with an adhesive (not shown in the drawings) covered by a protective strip 118, as seen in FIG. 7. The cushion 116 defines creases 120 extending generally parallel to the strap 114 for facilitating folding of the cushion 116 in a closed position, shown in FIGS. 8 and 9.

The strap 114 defines a deformable section 122 located substantially adjacent the biting section 112. For example, the deformable section 122 includes embossed grooves 124 substantially longitudinally spaced apart from each other and each extending substantially perpendicularly to the longitudinal axis of the strap 114 to facilitate stretching of the strap 114 to accommodate sensors having different dimensions while maintaining a suitable tension in the strap 114. The free end of the strap 114 is provided with detachable sections 126 defined by notches 128 to adjust the length of the strap 114.

In use, once the protective strip 118 is removed, the strap 114 is wrapped securely around the sensor and secured by the adhesive. Once the strap 114 is secured to the adhesive, the end sections of the cushioned biting section 112 are folded back until they meet. Two lines are sealed on both sides of the center of the cushion 116, which causes the joined end sections of the cushion 116 to form a centered biting tab, as seen in FIGS. 8 and 9.

Due to the fact that sensors are made in different widths and thicknesses, the detachable sections 126 are provided at the end of the strap 114 to allow the dental professional an easy way to tear off any extra strap portion.

For example, the strap 114 and the backing of the biting section 112, ie the portion of the biting section 112 that supports the cushion 116 which is secured thereto, is a 12 point vinyl which although durable, does not add significant bulk to the sensor when the bite wing 110 is attached thereto. The cushion 116 includes a foam, for example a soft vinyl foam approximately ⅛″ thick, (¼″ thick when combined both sides) that is welded to the vinyl backing.

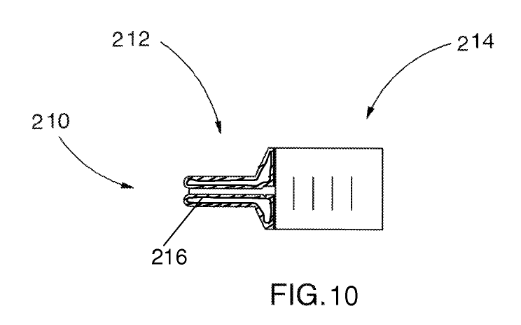

In some embodiments of the invention, as shown in FIG. 10, an alternative bite wing 210 includes a tension strap 214 similar to the tension strap 114 and an alternative biting section 212. The biting section 212 defines a cavity 216 filled with air or a any other suitable gas. The periphery of the cavity 216 is made out of a resistant material that can resist the bite of a human. In other words, the cushion 116 in these embodiments is replaced by a gas filled bladder that provides cushioning while providing adaptability to the bite of a wide range of teeth and jaw morphologies.

Although the present invention has been described hereinabove by way of preferred embodiments thereof, it can be modified, without departing from the spirit and nature of the subject invention as defined in the appended claims.

What is claimed is:

1. A positioning bite tab for a dental sensor, said positioning bite tab comprising: a central section and two wings extending substantially opposed to each other from said central section, each of said wings including a cushioned section, said cushioned section including a gas filled bladder.
